(19) United States
(12) Patent Application Publication
(10) Pub. No.: US 2016/0279359 A1
(43) Pub. Date: Sep. 29, 2016

(54) Inductive Breathing Mask and Respiratory Care System Thereof

(71) Applicant: TAICHUNG VETERANS GENERAL HOSPITAL, Taichung (TW)

(72) Inventors: PI-HUA CHANG, Taichung (TW); SHIH-HUNG LIN, Taichung (TW); CHUN-SHIH CHIN, Taichung (TW); YI-JEN CHUNG, Taichung (TW)

(21) Appl. No.: 15/054,116

(22) Filed: Feb. 25, 2016

(30) Foreign Application Priority Data
Feb. 25, 2015 (TW) 104105941

(51) Int. Cl. A61M 16/00; A61M 16/06

(52) U.S. Cl. CPC A61M 16/0003; A61M 16/0622; A61M 16/0051; A61M 2205/3344; A61M 2016/0027; A61M 2210/0606; A61M 2205/502

(57) ABSTRACT

An inductive breathing mask is provided, which comprises a covering shell, a mask pad, and a pressure inductive unit, wherein the pressure inductive unit comprising at least one pressure inductive part, and the pressure inductive part is disposed on the mask pad where corresponds to at least one of the user's nasion, left cheek beside nose, right cheek beside nose, and chin for detecting the pressure of the user's nasion, left cheek beside nose, right cheek beside nose, and chin generated by the mask pad. In addition, the inductive breathing mask of the present invention may further be integrated with other functional elements for constructing a respiratory care system having warning and/or monitoring functions.

### CROSS REFERENCE OF RELATED APPLICATION

This application claims the benefits of Taiwan Patent Application Serial Numbers 104105941, filed on Feb. 25, 2015, the subject matter of which is incorporated herein by reference.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

The present invention relates to an inductive breathing mask and a respiratory care system. More particularly, the present invention relates to a breathing mask and a respiratory care system wherein the compression level of the user's face may be detected thereby.

2. Description of Related Arts

The long-term bed-ridden patients who use a respirator and need hospital or home care are usually not able to express their feelings instantly. It is inevitable that those patients might suffer from decubitus caused by being in the same position for a long time because their professional caregivers cannot keep track of every single patient's conditions or needs. The main cause of decubitus is the ischemic necrosis of body soft tissue which occurs when a pressure is continuously applied onto a part of the body soft tissue beyond tolerance. And it might cause severe infections if the decubitus is not well-treated.

Intubation is applied in most of the former respiratory treatment which is likely to cause discomfort and many caring problems. However, if a breathing mask is used for replacing intubation, the patients wearing the breathing mask might suffer from the decubitus on their faces caused by the long-term compression from the breathing mask. Clinically, the tightness of the breathing mask is adjusted manually by the caregivers. Considering the different hand strength of different caregivers and unique facial bone and skin structure of the patients, the breathing mask might be too tight and causes early decubitus on the patient's face due to the excessive pressure. Even if the contact pressure of the breathing mask is adjusted to a preferable value, the excessive pressure from the breathing mask may still be applied to the patient's face due to their movement and causing decubitus. In this case, if the caregiver fails to readjust the breathing mask immediately by knowing the excessive pressure of the breathing mask, the decubitus will become more severe.

In view of the decubitus problems caused by the conventional breathing mask, it is desirable to provide a respiratory care system which is able to detect the compression level of the patient's face for helping the caregivers to avoid the incidence of decubitus caused by the excessive pressure of the breathing mask.

### SUMMARY OF THE PRESENT INVENTION

The object of the present invention is to provide an inductive breathing mask which is able to detect the contact pressure between the user's face and the breathing mask to overcome the tightness issue caused by the improper manual adjustment and to reduce the incidence of decubitus caused by the breathing mask. Accordingly, the inductive breathing mask is beneficial to hospital care or home care.

To achieve the object, the inductive breathing mask of the present invention comprises: a covering shell having an inner concave surface and a corresponding outer surface, wherein a chamber having an opening edge is defined by the inner concave surface, and an intake portion connecting to the chamber is disposed on the outer surface; a mask pad disposed along the opening edge of the covering shell and being placed on a user's face, the mask pad has a cyclic structure formed by a bottom portion, a first lateral portion, and a second lateral portion, wherein the bottom portion corresponds to the user's chin, the first lateral portion and the second lateral portion corresponds to the user's left cheek and right cheek respectively, and an intersection of the first lateral portion and the second lateral portion corresponds to the users' nasion; and a pressure inductive unit comprising at least one pressure inductive part, wherein the pressure inductive part is disposed on the mask pad where corresponds to at least one of the user's nasion, left cheek beside nose, right cheek beside nose, and chin.

The pressure inductive unit of the inductive breathing mask of the present invention may detect the compression level of the one or multiple locations of the user's face by single or multiple pressure inductive parts so that the caregivers may adjust the tightness of the mask based on the detection results. The number of the pressure inductive parts may be decided based on practical needs. For example, a four-point detection method is applied in a preferred embodiment of the present invention, wherein the pressure inductive unit consists a plurality of pressure inductive parts which are disposed correspondingly to the user's nasion (the nasal bridge between two eyes), left cheek beside nose, right cheek beside nose, and chin where are prone to suffer from decubitus. Furthermore, additional pressure inductive parts may be disposed correspondingly in other locations of the user's face when needed. The pressure inductive unit may be any types of pressure sensors for detecting pressure such as resistive pressure sensing unit or capacitive sensing unit, wherein the pressure inductive unit is preferably a flexible thin-film pressure sensor with a membrane-like pressure inductive parts. Accordingly, the thinned pressure inductive parts may bend and deform to comply with the user's face and the pattern of the mask pad so that leakage caused by the pressure inductive parts may be avoided and the sealing property may be maintained when these pressure inductive parts are disposed between the mask pad and the user's face.

In one embodiment of the present invention, the shape and the size of the pressure inductive parts of the pressure inductive unit are not particularly limited and may be altered based on practical needs. For example, according to one preferred embodiment of the present invention, the shape of the pressure inductive part may be a circle with 1 cm diameter, or may be a square, a rectangle, or other shapes which are not limited hereinto. Preferably, the pressure inductive parts are detachably disposed on the mask pad and facing the user's face so that the pressure inductive unit or other elements of the breathing mask may be replaced individually when needed instead of discarding the whole breathing mask. Here, the method for binding the pressure inductive parts of the pressure inductive unit and the mask pad is not particularly limited as long as the pressure inductive parts of the pressure inductive unit are able to detect the contact pressure between the breathing mask and the user's face. The pressure inductive parts of the pressure inductive unit may be embedded in the mask pad, for instance, a receiving bag or a receiving recess may be formed on a side of the mask pad which faces the user's face, and the pressure inductive parts may be accommodated in the receiving bag or be embedded in the receiving recess, wherein the exposed portions of the pressure inductive parts may further be covered by separating layers, which may be attached to the mask pad for fixing the disposed location of the pressure inductive part and avoiding the direct contact between the exposed portions of the pressure inductive parts and the user's face. Alternatively, the pressure inductive parts may be partially embedded in the mask pad with some portions of the pressure inductive parts being located at an outer of the mask pad, that is, a portion of the pressure inductive parts may be embedded in the receiving recess, and another portion of the pressure inductive parts protrude out of the receiving recess. Similarly, the protruded portion of the pressure inductive parts may be covered by separating layers for fixation and separation. Also, the pressure inductive parts may be disposed on the mask pad and be fixed between the mask pad and the user's face by the separating layers. The separating layers are utilized to fix the disposing location of the pressure inductive parts and to avoid the direct contact between the pressure inductive parts and the user's face. Therefore, the separating layers are not particularly limited and can be any types of adhesive tapes that being adaptable for skin such as bandages, breathable tapes, artificial skin, or medical patches. In addition, when the mask pad is a single-layered pad, the pressure inductive parts may be directly disposed on one side of the single-layered pad which faces the user's face; when the mask pad is a multi-layered pad with a bottom pad and protective layers, the pressure inductive parts may be disposed at the protective layers because the user's face contacts with the protective layers that sleeved to the bottom pad. The protective layers may improve the sealing property of the breathing mask and may be disassembled for cleaning or replacement. Because the pressure inductive unit is detachably disposed on the protective layers, the pressure inductive unit may be disassembled when the protective layers are disassembled for cleaning or replacement; or the pressure inductive unit may be replaced individually when the pressure inductive unit is malfunction because the pressure inductive unit is detachably disposed on the protective layers.

Accordingly, the inductive breathing mask of the present invention may detect the contact pressure between the mask pad and the user's face through the pressure inductive unit. The pressure inductive unit may further be integrated with other functional elements so that the caregivers may monitor the compression level of the user's face instantly. Therefore, the present invention further provides a respiratory care system comprising the aforementioned inductive breathing mask, wherein the pressure inductive unit of the pressure inductive breathing mask is utilized for detecting the pressure generated on the user's nasion, left cheek beside nose, right cheek beside nose, and chin against the mask pad and generates a pressure signal; a processing unit which is connected to the pressure inductive unit for receiving the pressure signal and generates a display signal; and a displaying unit which is connected to the processing unit for receiving the display signal and displays a pressurized information of at least one of the user's nasion, left cheek beside nose, right cheek beside nose, and chin according to the display signal.

The respiratory care system of the present invention further comprises a warning unit which is connected to the processing unit for receiving a warning signal generated from the processing unit and generates a warning information, wherein the processing unit generates the warning signal when at least one of the pressurized signal the pressure signal surpasses a pressure set value. Accordingly, when the breathing mask is too tight, the warning unit may instantly remind the caregivers to adjust the tightness of the mask for overcoming the problem of tight mask caused by the improper manual adjustment. In addition, the respiratory care system of the present invention may further comprises a monitoring unit which is connected to the processing unit for receiving an notification signal generated from the processing unit and generates a notification message, wherein the processing unit generates the notification signal when the pressurized signal surpasses a pressure set value and a pressurized time set value. Accordingly, even if the caregiver is unable to provide care for patients on site, the caregivers may immediately be informed about the abnormal conditions when the breathing mask dislocates due to patient's movement which causes the overpressure for a period of time in order to achieve the real-time monitoring effect.

Refer to the respiratory care system of the present invention, the displaying unit may be any devices that are able to display (such as a screen) so that the compression level of the breathing mask when adjusting the tightness of the breathing mask may be monitored by the caregivers. The processing unit may be any devices having the operation or control functions (such as a central processor, or programmable microprocessor) for controlling each unit.

Refer to the respiratory care system of the present invention, the pressure set value and the pressurized time set value may be pre-set by a setting unit, that is, the respiratory care system of the present invention further comprises a setting unit connected to the processing unit for setting the pressure set value and/or the pressurized time set value. The setting unit may be any devices having the setting function (such as a keyboard, a touchscreen, or a remote controller) which may pre-set at least one pressure set value and at least one pressurized time set value. These set values are preferably lower than the critical value for causing decubitus to prevent the conditions that cause decubitus. For example, the pressure set value may be pre-set to 32 mmHg and the pressurized time set value may be pre-set to 2 hours. The warning unit may be driven to generate the warning information such as vibration, light, sound, or text for reminding the caregivers when any one of the pressure inductive parts of the pressure inductive unit detects a pressure signal that surpasses the pressure set value so that the caregivers may instantly adjust the breathing mask. Simultaneously, the processing unit may generate an notification signal to the monitoring unit (such as mobile phone, tablet PCs, notebook computers, personal computers, or personal digital assistants) and the monitoring unit is driven to generate a notification message such as vibration, light, sound, or text for reminding the caregivers when any one of the pressure inductive parts of the pressure inductive unit detects a pressure signal that surpasses the pressure set value and the pressurized time set value so that the caregivers may instantly solve the problems of overpressure caused by the dislocation of the breathing mask. In addition, the pressure set value and the pressurized time set value corresponds to each of the pressure inductive parts may be pre-set separately, wherein the pressure set values and the pressurized time set values thereof may be the same or different. The processing unit will generate the warning signal when the pressures of each of the pressure inductive parts surpass the corresponding pressure set values. Simultaneously, the processing unit will generate the notification signal when the pressure and the pressurized time of each of the pressure inductive parts surpass the corresponding pressure set values and the corresponding pressurized time set values.

Refer to the respiratory care system of the present invention, the signal connections between each of the units may be linked by cables. Alternatively, the respiratory care system may further comprise a wireless communication unit so that the signal connections between each of the units may be linked by wireless transmission technology such as infrared, Bluetooth, Z-Wave, NFC, Zigbee, or Wi-fi. The monitoring unit is preferably connected to the processing unit through the wireless transmission, which is beneficial to the caregivers for remotely keeping track of the compression level of the user's face.

In summary, the contact pressure between the user's face and the breathing mask may be detected by the pressure inductive unit; therefore, the caregivers may overcome the problem of tight mask caused by the improper manual adjustment immediately based on the detected compression level. In the meantime, when the caregivers cannot take care of the patients on site, the abnormal conditions of the breathing mask can still be informed through the monitoring unit for reducing the incidence of decubitus caused by the long term compression. Accordingly, the inductive breathing mask and the respiratory care system of the present invention provide a more humane and accurate way of applying the breathing mask with precise compression level against the user's face; reduce the risk of decubitus on patients' face, and reduce the routine work of checking the mask for the caregivers. Therefore, the present invention is beneficial to home and hospital care and can serve as the basis of the future telemedicine.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of an inductive breathing mask of an embodiment of the present invention;

FIG. 2 is a front view of the inductive breathing mask of an embodiment of the present invention;

FIG. 3 is a rear view of the inductive breathing mask of an embodiment of the present invention;

FIG. 4 is a wearing schematic diagram of the inductive breathing mask of an embodiment of the present invention;

FIG. 5 and FIG. 6 are a first arranging schematic diagram of the pressure inductive unit of an embodiment of the present invention;

FIG. 7 is a second arranging schematic diagram of the pressure inductive unit of an embodiment of the present invention;

FIG. 8 is a third arranging schematic diagram of the pressure inductive unit of an embodiment of the present invention;

FIG. 9 is a fourth arranging schematic diagram of the pressure inductive unit of an embodiment of the present invention;

FIG. 10 is the first arranging schematic diagram of the pressure inductive unit of another embodiment of the present invention;

FIG. 11 is the second arranging schematic diagram of the pressure inductive unit of another embodiment of the present invention;

FIG. 12 is the third arranging schematic diagram of the pressure inductive unit of another embodiment of the present invention;

FIG. 13 is the fourth arranging schematic diagram of the pressure inductive unit of another embodiment of the present invention;

FIG. 14 is a block diagram of the respiratory care system of an embodiment of the present invention; and

FIG. 15 is a block diagram of the respiratory care system of another embodiment of the present invention.

### DETAILED DESCRIPTION OF THE PREFERRED EMBODIMENT

Hereafter, examples will be provided to illustrate the embodiments of the present invention. Advantages and effects of the invention will become more apparent from the disclosure of the present invention. It should be noted that these accompanying figures are-simplified and illustrative. The quantity, shape and size of components shown in the figures may be modified according to practical conditions, and the arrangement of components may be more complex. Other various aspects also may be practiced or applied in the invention, and various modifications and variations can be made without departing from the spirit of the invention based on various concepts and applications.

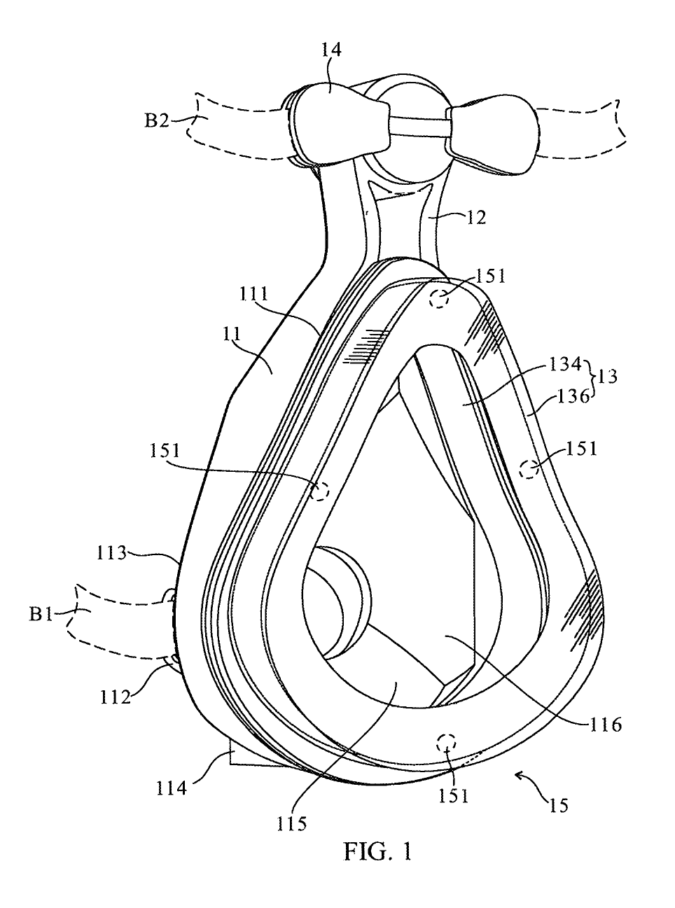

With reference to FIG. 1, there is shown a perspective view of the inductive breathing mask 10 of an embodiment of the present invention. The breathing mask 10 of the present embodiment mainly comprises a covering shell 11, a mask pad 13, and a pressure inductive unit 15, wherein the mask pad 13 is disposed along an opening edge 111 of the covering shell 11 and is placed on a user's face, and the pressure inductive unit 15 has a plurality of pressure inductive parts 151 which are disposed on the mask pad 13 for detecting the pressure against the user's face caused by the breathing mask 10.

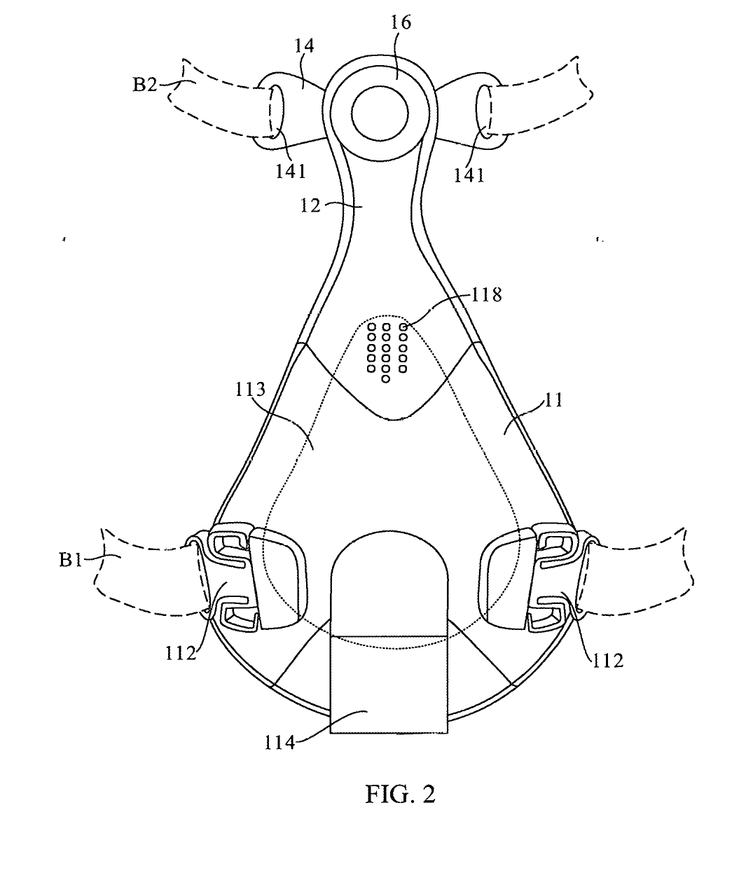

Please refer to FIG. 2, which shows the front view of the inductive breathing mask 10 of the present embodiment. In detail, the inductive breathing mask 10 binds with the fixing band B1 through the binding joint 112 wherein the fixing band B1 is utilized for wearing the inductive breathing mask 10 onto the user's face. A concave surface 15 of the covering shell 11 faces toward the user's nose and mouth and defines a chamber 116, wherein the chamber 116 is connected to an intake portion 114 which is disposed on an outer surface 113 of the covering shell 11 for inputting gases coming from an external inflator (not shown) into the chamber 116. Simultaneously, the carbon dioxide exhaled from the user may be discharged from the chamber 116 through a plurality of vents 118 disposed on the covering shell 11. Further, the inductive breathing mask 10 further comprises a supporting element 12 which extends from an upper end of the covering shell 11 and connects with an abutting element 14. The supporting element 12 binds to the fixing band B2 with the binding joints 141 at two ends of the abutting element 14 so that the fixing band B2 may be fastened to the user's head and the abutting element 14 may be fixed on the user's forehead. The adhesion between the abutting element 14 and the user's forehead may be adjusted by an adjusting element 16 for ensuring the coverage of the inductive breathing mask 10.

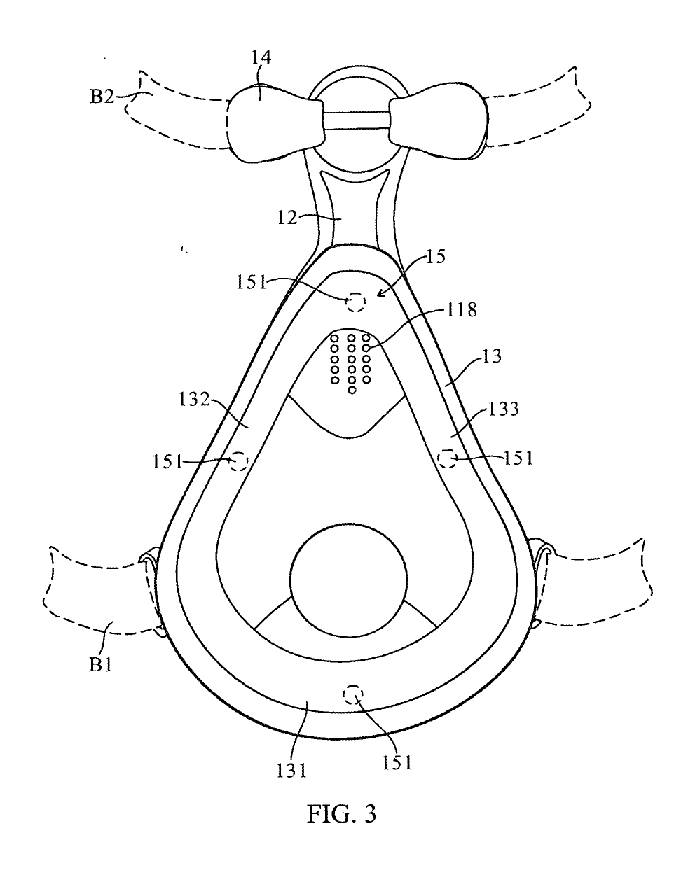

Please refer to FIG. 3, which shows the rear view of the inductive breathing mask 10 of the present embodiment. As shown in FIG. 1 and FIG. 3, the mask pad 13 of the inductive breathing mask 10 of the present embodiment has a cyclic structure formed by a bottom portion 131, a first lateral portion 132, and a second lateral portion 133, and has a double-layered structure having a bottom pad 134 and a protective layer 136, wherein the protective layer 136 is sleeved to the bottom pad 134. The pressure inductive parts 151 of the pressure inductive unit 15 are disposed on the protective layer 136 of the mask pad 13 to detect the contact pressure caused by adhering the mask pad 13 to the user's face. Accordingly, as shown in FIG. 4, the bottom portion 131 corresponds to the user's chin, the first lateral portion 132 and the second lateral portion 133 correspond to the user's left cheek and right cheek respectively, and an intersection of the first lateral portion and the second lateral portion corresponds to the user's nasion when the inductive breathing mask 10 is placed on the user's face. Simultaneously, the pressure inductive parts 151, which are disposed at the mask pad 13, corresponds to the user's nasion, left cheek beside nose, right cheek beside nose, and central chin respectively for keeping track of the compression condition of user's face precisely through multi-points detection. In the present embodiment, the pressure inductive parts of the pressure inductive unit are designed as a circular pressure inductive part with 1 cm diameter, and the pressure inductive unit is illustrated as a demonstration of four-point detection. The number, shape, and size of the pressure inductive parts are not limited to the demonstration of the present embodiment and can be altered based on practical needs. For example, the shape of the pressure inductive parts may be square or rectangle which is not limited to the illustrated circular shape. Also, the number of the pressure inductive part may be one for single-point detection which is not limited to the multi-point detection that illustrated in the present embodiment. Alternatively, additional pressure inductive parts may be disposed at other locations that correspond to the user's face when needed.

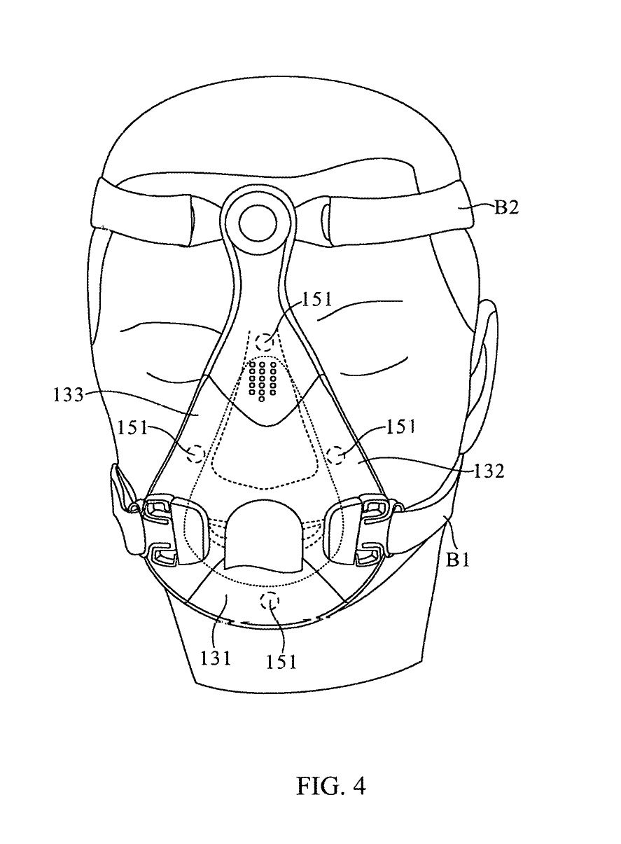

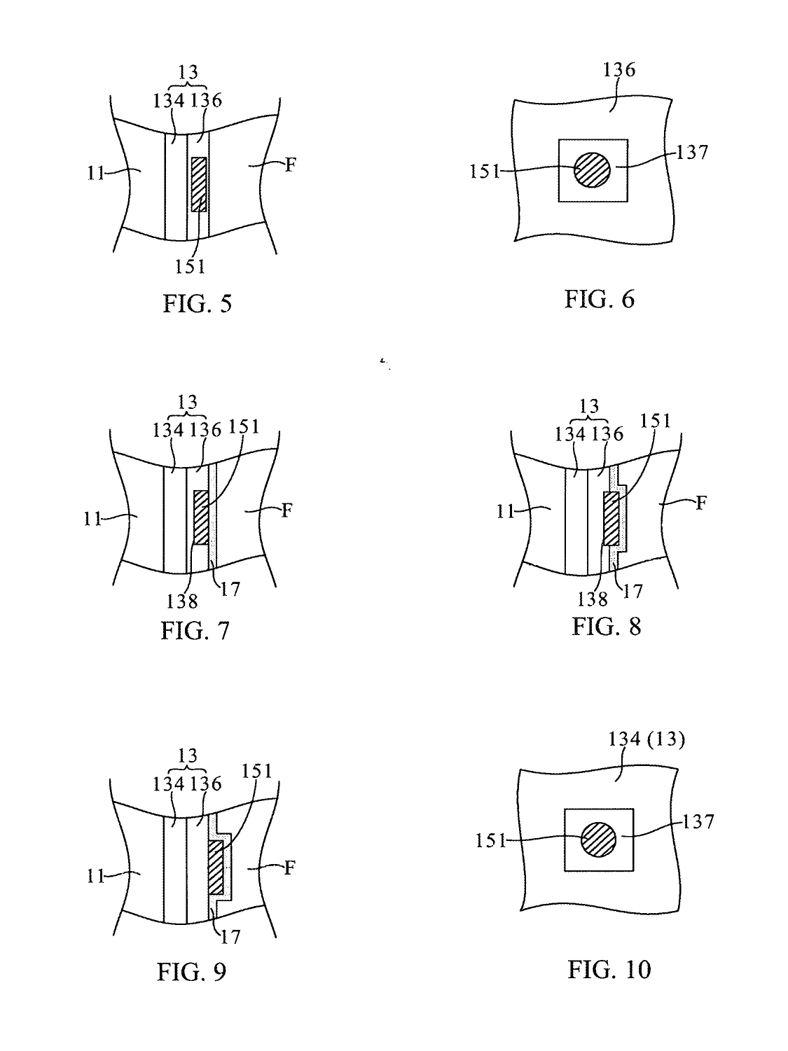

Please refer to FIG. 5 to FIG. 9 which show the arrangement of the pressure inductive parts of the pressure inductive unit. As illustrated in FIG. 5, the pressure inductive parts 151 are accommodated at one side of the mask pad 13 which faces the user's face F for detecting the contact pressure between the mask pad 13 and the user's face F. In detail, the pressure inductive parts 151 may be detachably disposed in the protective layer 136 of the mask pad 13. Please refer to FIG. 6 simultaneously, a receiving bag 137 may be disposed on the protective layer 136 for accommodating the pressure inductive parts 151. Alternatively, as illustrated in FIG. 7, a receiving recess 138 may be disposed on the side of the protective layer 136 which faces the user's face F for the pressure inductive parts 151 embedded therein. The exposed portions of the pressure inductive parts 151 may be covered by separating layers 17, wherein the separating layers 17 may attach to the protective layer 136 for fixing the disposed location of the pressure inductive parts 151 and avoiding the direct contact between the pressure inductive parts 151 and the user's face F. Alternatively, as illustrated in FIG. 8, only a portion of the pressure inductive parts 151 are embedded in the receiving recess 138, and another portion thereof protrude out of the receiving recess 138 and being covered by the separating layers for fixation. Also, the pressure inductive parts 151 may be fixed to the protective layer 136 of the mask pad 13 by the separating layers directly so that the pressure inductive parts 151 are disposed between the mask pad 13 and the user's face F. In the present embodiment, the thin-film resistive type pressure inductive unit having membrane-like pressure inductive parts 151 is used, wherein the pressure inductive parts 151 are flexible so that the gas leakage caused by poor fitting adhesion between the breathing mask and the user's face may be avoided. In addition, the separating layer 17 can be any adhesive tapes that adaptable for skin such as bandages, breathable tapes, artificial skin, or medical patches.

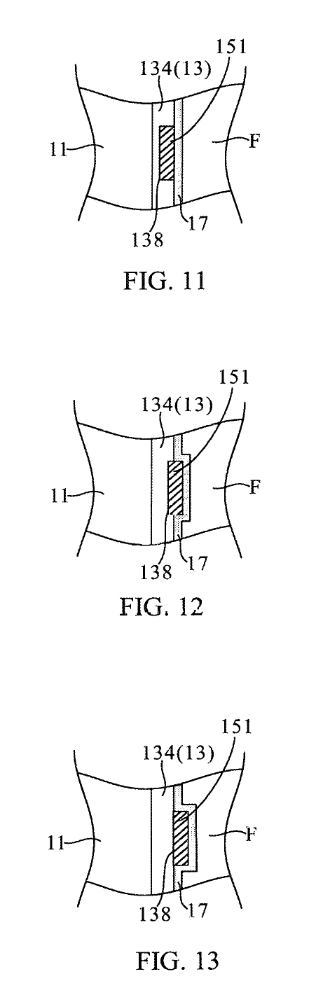

The mask pad 13 of the inductive breathing mask 10 may be a single-layered pad, wherein the mask pad 13 contacts to the user's face F directly with the bottom pad 134 thereof. Accordingly, as illustrated in FIG. 10, a receiving bag 137 may be disposed on the bottom pad 134 for receiving the pressure inductive parts 151. Or, as illustrated in FIG. 11, a receiving recess 138 may be disposed on one side of the bottom pad 134 which faces the user's face F for receiving the pressure inductive parts 151. A separating layer 17 may be utilized to cover the pressure inductive parts 151, and may be attached to the bottom pad 134 for fixation and separation. Alternatively, as illustrated in FIG. 12, only a portion of the pressure inductive parts 151 are embedded in the receiving recess 138. Another portion of the pressure inductive parts 151 protrude out of the receiving recess 138 and the disposed locations are fixed by the separating layer 17. Also, as illustrated in FIG. 13, the pressure inductive parts 151 may be fixed directly to the bottom pad 134 by the separating layer 17 so that the pressure inductive parts 151 are disposed between the mask pad 13 and the user's face F.

According to the aforementioned arrangement of the pressure inductive parts, the inductive breathing mask of the present embodiment may detect the compression conditions of the user's nasion, left cheek beside nose, right cheek beside nose, and central chin through the pressure inductive unit. The inductive breathing mask may further be integrated with other functional elements so that the caregivers may monitor the compression level of the user's face instantly.

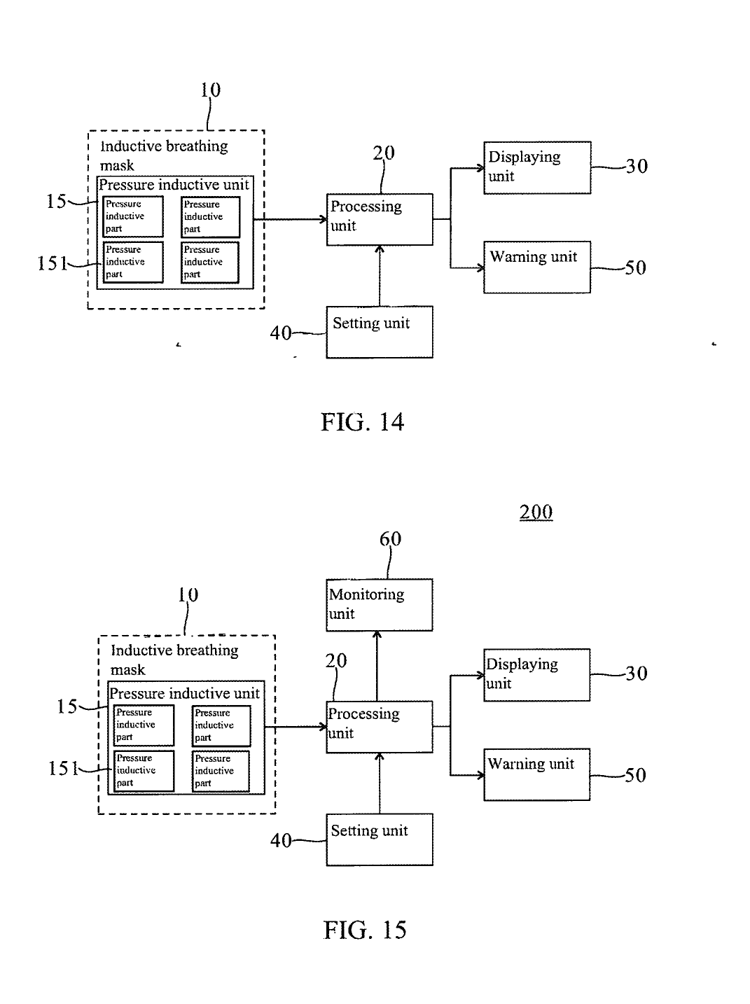

Please refer to FIG. 14, there is shown a block diagram of the respiratory care system of an embodiment of the present invention. As illustrated in FIG. 14, the respiratory care system 100 of the present embodiment comprises an inductive breathing mask 10, a processing unit 20, a displaying unit 30, a setting unit 40, and a warning unit 50. The breathing mask 10, as described above, has a pressure inductive unit 15, wherein the pressure inductive unit 15 consists of a plurality of pressure inductive parts 151 and is connected to the processing unit 20. The processing unit 20 is further connected to the displaying unit 30, the setting unit 40, and the warning unit 50.

Accordingly, when the inductive breathing mask 10 is placed and covers the user's mouth and nose, the pressure generated on the user's nasion, left cheek beside nose, right cheek beside nose, and central chin by the inductive breathing mask 10 may be detected by the pressure inductive unit 15. Afterwards, at least one pressure signals generated by the pressure inductive unit 15 is then be sent to the processing unit 20 (such as a central processor, or other programmable microprocessor), and the processing unit 20 generates a display signal right after receiving the pressure signal. Based on the display signal, the displaying unit 30 (such as screen) will display the pressurized information of the user's nasion, left cheek beside nose, right cheek beside nose, and central chin after receiving the display signal. According to the pressurized information displayed by the displaying unit 30, the contact pressure between the breathing mask and the user's face may be provided directly to the caregivers. Hence, the caregivers may adjust the tightness of the breathing mask properly to avoid the incidence of decubitus caused by the tight breathing mask. In order to achieve the object of instant notification, the caregivers may pre-set a pressure set value using the setting unit 40 (such as a keyboard, a touchscreen, or a remote controller), therefore, the processing unit 20 may generate a warning signal when the pressure signal surpasses a pressure set value. The warning unit 50 will be driven to generate the warning information such as vibration, light, sound, or text after receiving the warning signal for reminding the caregivers to adjust the breathing mask. In the present embodiment, the pressure set value is 32 mmHg, and if the pressure signal detected by any one of the pressure inductive parts 151 of the pressure inductive unit 15 surpasses the pressure set value, the processing unit 20 will generate the warning signal. Alternatively, the pressure set values correspond to each of the pressure inductive parts 151 may be pre-set separately, wherein the pressure set values thereof may be the same or different according to practical needs. Also, if the pressure signal detected by anyone pressure inductive part 151 surpasses its pressure set value, the processing unit 20 will generate the warning signal.

Furthermore, after the contact pressure of the breathing mask is adjusted to a preferable value, the dislocation of the breathing mask may still happen due to the movements of the user. In order to prevent the decubitus that incidents when the skin is pressurized for too long, the respiratory care system may further be combined with the concept of monitoring to assist the caregivers in monitoring the pressurized condition of the user's face. Accordingly, please refer to FIG. 15, there is shown a block diagram of a respiratory care system 200 of another embodiment, wherein the respiratory care system 200 further comprises a monitoring unit 60 which is connected to the processing unit 20 for receiving an notification signal and generating a notification message.

In detail, the caregivers may pre-set a pressure set value and a pressurized time set value using the setting unit so that the processing unit 20 may generate an notification signal when the pressure signal surpasses a pressure set value and a pressurized time set value. The notification signal will be sent to the monitoring unit 60 (such as mobile phone, tablet PCs, notebook computers, personal computers, or personal digital assistants) and the monitoring unit is driven to generate a notification message such as vibration, light, sound, or text for reminding the caregivers to instantly solve the problems of overpressure caused by the dislocation of the breathing mask. In the present embodiment, the pressure set value is 32 mmHg and the pressure set value is 2 hours. If the pressure signal detected by any one of the pressure inductive parts 151 of the pressure inductive unit 15 surpasses the pressure set value or the pressurized time set value, the processing unit will generate the notification signal. Alternatively, as described above, the pressure set values and the pressurized time set values correspond to each of the pressure inductive parts 151 may be pre-set separately, wherein the pressure set values and the pressurized time set values thereof may be the same or different according to practical needs. Also, if the pressure signal detected by anyone pressure inductive part 151 surpasses its pressure set value and it's pressurized time set value, the processing unit 20 will generate the notification signal.

In the aforementioned embodiment, the signal connections between each of the units may be linked by cables. Alternatively, the respiratory care system may further comprise a wireless communication unit so that the signal connections between each of the units may be linked by wireless transmission technology (such as infrared, Bluetooth, Z-Wave, NFC, Zigbee, or Wi-fi). The monitoring unit 60 is preferably connected to the processing unit 20 through the wireless transmission, which is beneficial to the caregivers for remotely keeping track of the compression level of the user's face. In addition, the pressure inductive unit is not limited to combine with the breathing mask that described in the former embodiment, that is, various breathing masks (such as the breathing mask without supporting element and abutting element, of the breathing mask having a the supporting element but the abutting element is not adjustable) may be combined with the pressure inductive unit to accomplish the inductive breathing mask of the present invention.

In summary, the present invention integrates the pressure inductive unit into the breathing mask and further integrates other functional units to accomplish a respiratory care system with displaying, warning and/or monitoring functions. Accordingly, the respiratory care system of the present invention may reduce the incidence of decubitus caused by the breathing mask and is beneficial to hospital care or home care for patients with respiratory treatment.

It should be noted that the aforementioned embodiments are only exemplified for descriptive conveniences, the scope of the present invention should be subjected to the following claims, and not limited to the aforementioned embodiments.

1. An inductive breathing mask, comprising:
a covering shell having an inner concave surface and a corresponding outer surface, wherein a chamber having an opening edge is defined by the inner concave surface, and an intake portion connecting to the chamber is disposed on the outer surface;
a mask pad disposed along the opening edge of the covering shell and being placed on a user's face, the mask pad has a cyclic structure formed by a bottom portion, a first lateral portion, and a second lateral portion, wherein the bottom portion corresponds to the user's chin, the first lateral portion and the second lateral portion correspond to the user's left cheek and right cheek respectively, and an intersection of the first lateral portion and the second lateral portion corresponds to the user's nasion; and
a pressure inductive unit comprising at least one pressure inductive part, and the pressure inductive part is disposed on the mask pad where corresponds to at least one of the user's nasion, left cheek beside nose, right cheek beside nose, and chin.

2. The inductive breathing mask as claimed in claim 1, wherein the pressure inductive unit comprising a plurality of pressure inductive parts that correspond to the user's nasion, left cheek beside nose, right cheek beside nose, and chin.

3. The inductive breathing mask as claimed in claim 1, wherein the pressure inductive part is accommodated in the mask pad.

4. The inductive breathing mask as claimed in claim 1, wherein a portion of the pressure inductive part is embedded in the mask pad, and another portion of the pressure inductive part is located at an outer of the mask pad.

5. The inductive breathing mask as claimed in claim 1, wherein the pressure inductive part is disposed on the mask pad.

6. The inductive breathing mask as claimed in claim 4, further comprising a separating layer covering the pressure inductive part and attached to the mask pad.

7. The inductive breathing mask as claimed in claim 1, wherein the pressure inductive unit is a thin-film pressure inductive unit.

8. The inductive breathing mask as claimed in claim 7, wherein the pressure inductive unit is a thin-film resistive type pressure inductive unit.

9. A respiratory care system comprising:
an inductive breathing mask as claimed in claim 1, wherein the pressure inductive unit is utilized for detecting pressure generated from at least one of the user's nasion, left cheek beside nose, right cheek beside nose, and chin against the mask pad and generating at least one pressure signal;
a processing unit which is connected to the pressure inductive unit for receiving the pressure signal and generates a display signal; and
a displaying unit which is connected to the processing unit for receiving the display signal and displays a pressurized information of at least one of the user's nasion, left cheek beside nose, right cheek beside nose, and chin according to the display signal.

10. The respiratory care system as claimed in claim 9 further comprising a warning unit which is connected to the processing unit for receiving a warning signal generated from the processing unit and generates a warning information.

11. The respiratory care system as claimed in claim 10, wherein the processing unit generates the warning signal when the pressure signal surpasses a pressure set value.

12. The respiratory care system as claimed in claim 11, further comprising a setting unit which is connected to the processing unit for setting the pressure set value.

13. The respiratory care system as claimed in claim 9, further comprising a monitoring unit which is connected to the processing unit for receiving an notification signal generated from the processing unit and generates a notification message.

14. The respiratory care system as claimed in claim 13, wherein the processing unit generates the notification signal when the pressure signal surpasses a pressure set value and a pressurized time set value.

15. The respiratory care system as claimed in claim 14, further comprising a setting unit which is connected to the processing unit for setting the pressure set value and the pressurized time set value.

16. The inductive breathing mask as claimed in claims 5, further comprising a separating layer covering the pressure inductive part and attached to the mask pad.

17. The respiratory care system as claimed in claims 10, further comprising a monitoring unit which is connected to the processing unit for receiving an notification signal generated from the processing unit and generates a notification message.

18. The respiratory care system as claimed in claim 17, wherein the processing unit generates the notification signal when the pressure signal surpasses a pressure set value and a pressurized time set value.

19. The respiratory care system as claimed in claim 18, further comprising a setting unit which is connected to the processing unit for setting the pressure set value and the pressurized time set value.
